(19) United States
(12) Patent Application Publication
(10) Pub. No.: US 2025/0073419 A1
(43) Pub. Date: Mar. 6, 2025

(54) Spring-Loaded Valve Urine Backflow Prevention Device and Method of Use

(71) Applicant: John Plehinger, Denver, CO (US)

(72) Inventor: John Plehinger, Denver, CO (US)

(21) Appl. No.: 18/806,225

(22) Filed: Aug. 15, 2024

Related U.S. Application Data
(60) Provisional application No. 63/579,658, filed on Aug. 30, 2023.

(51) Int. Cl. A61M 25/00; A61M 39/24

(52) U.S. Cl. CPC A61M 25/0017; A61M 39/24; A61M 2039/2406

(57) ABSTRACT

A urine backflow prevention device designed for use with catheters to prevent expelled urine from reentering the body is disclosed. The device comprises a catheter tube with an integrated housing containing a valve and a spring mechanism. The valve is made of medical-grade plastic or rubber and is configured to open in response to urine pressure (i.e., pressure sensitive) and close when urine flow ceases. The spring is made of corrosion-resistant metal and provides the necessary resistance to keep the valve closed in the absence of urine flow. This mechanism effectively prevents backflow, thereby reducing the risk of urinary tract infections. The device is positioned near the proximal end of the catheter tube and is suitable for use in various medical applications where maintaining a sterile environment is critical.

### CROSS-REFERENCE TO RELATED APPLICATION

The present application claims priority to, and the benefit of, U.S. Provisional Application No. 63/579,658 which was filed on Aug. 30, 2023 and is incorporated herein by reference in its entirety.

### FIELD OF THE INVENTION

The present invention generally relates to the field of medical devices, particularly those used in urinary management. More specifically, the present invention pertains to a urine backflow prevention device designed for use with catheter tubes. The device integrates a spring-loaded valve mechanism within the catheter to prevent expelled urine from reentering the body. The device operates by opening the valve in response to urine pressure (i.e., pressure sensitive) and closing it immediately after the flow ceases, thereby providing an effective barrier against backflow. The device enhances patient safety by reducing the risk of urinary tract infections (UTIs) and ensuring the sterility of the urinary tract. Accordingly, the present disclosure makes specific reference thereto. Nonetheless, it is to be appreciated that aspects of the present invention are also equally applicable to other like applications, devices, and methods of manufacture.

### BACKGROUND

By way of background, catheter tubes are widely used in medical settings to drain urine from the bladder, especially in patients who are unable to urinate naturally due to various medical conditions or during and after surgical procedures. The catheter tubes are connected to urine collection bags, which store the expelled urine for disposal. However, a significant problem associated with the use of catheter tubes and urine collection bags is the potential for urine backflow.

Urine backflow occurs when expelled urine flows back into the bladder or urinary tract from the collection bag or catheter tube. This backflow can lead to severe infections, such as urinary tract infections (UTIs), due to the introduction of bacteria from the external environment back into the body. Additionally, urine backflow can create uncomfortable pressure within the bladder, exacerbating the patient's discomfort and potentially leading to further complications. To prevent these issues, it is crucial to ensure that urine, once expelled from the bladder, cannot reenter the body. Accordingly, individuals desire urine backflow prevention device that can be integrated into catheter tubes.

Therefore, there exists a long-felt need in the art for an effective solution to prevent urine backflow in catheterized patients, which can lead to serious complications such as urinary tract infections (UTIs) and increased patient discomfort. There is also a long-felt need in the art for a reliable device that can be integrated into catheter systems to ensure that once urine is expelled from the bladder, it does not reenter the body, thereby maintaining the sterility of the urinary tract. Additionally, there is a long-felt need in the art for a catheter device that is simple to use, durable, and does not require frequent maintenance or replacement. Moreover, there is a long-felt need in the art for a device that can be easily sterilized or made disposable to further reduce the risk of infection. Furthermore, there is a long-felt need in the art for a urine backflow prevention system that can be used in a variety of medical settings, providing consistent performance and improving patient outcomes.

The subject matter disclosed and claimed herein, in one embodiment thereof, comprises a urine backflow prevention device designed to be integrated into catheter tubes used for draining urine from the bladder. The device includes a spring-loaded valve mechanism housed within the catheter tube. The valve is configured to open in response to urine pressure (i.e., pressure sensitive), enabling the urine to flow out of the catheter, and to close immediately when urine flow ceases, thereby preventing any backflow. The valve is made from medical-grade materials that are resistant to the chemical properties of urine, ensuring long-term durability and reliability. The spring mechanism is designed to provide the necessary resistance to keep the valve closed when there is no urine flow, yet is sensitive enough to open when urine is expelled from the bladder.

In this manner, the urine backflow prevention device of the present invention accomplishes all of the foregoing objectives and provides a novel solution for managing urine flow in catheterized patients. The device integrates with existing catheter systems and provides an effective barrier against urine backflow. The spring-loaded valve mechanism is housed within the catheter tube with the valve opening in response to urine pressure (i.e., pressure sensitive) and closing immediately after flow ceases. The device prevents any expelled urine from reentering the body, significantly reducing the risk of infections and ensuring patient comfort. The device is constructed from durable, biocompatible materials, and may include additional features such as a filter to prevent debris from entering the catheter. The device not only improves patient safety but also enhances the overall efficiency and reliability of catheter systems in various medical settings.

### SUMMARY OF THE INVENTION

The following presents a simplified summary in order to provide a basic understanding of some aspects of the disclosed innovation. This summary is not an extensive overview, and it is not intended to identify key/critical elements or to delineare the scope thereof. Its sole purpose is to present some general concepts in a simplified form as a prelude to the more detailed description that is presented later.

The subject matter disclosed and claimed herein, in one embodiment thereof, comprises a urine backflow prevention device. The device comprises a catheter tube adapted to be inserted into a body to drain urine from a bladder, a housing is integrated within the catheter tube, a valve is disposed within the housing, wherein the valve is configured to open in response to urine pressure (i.e., pressure sensitive) to enable urine to flow out of the catheter tube, and to close when urine flow ceases, and a spring is disposed within the housing and connected to the valve, the spring being calibrated to provide resistance sufficient to keep the valve closed in the absence of urine flow and to enable the valve to open when urine is expelled.

In yet another embodiment, a catheter system for preventing urine backflow is disclosed. The system comprises a catheter tube having a proximal end adapted for insertion into a bladder and a distal end for connection to a urine collection bag, a backflow prevention mechanism is disposed within the catheter tube near the proximal end, the backflow prevention mechanism comprises a valve made of medical-grade material that is resistant to the chemical properties of urine and a spring is made of corrosion-resistant metal, wherein the spring is operatively connected to the valve and is configured to maintain the valve in a closed position in the absence of urine flow and to enable the valve to open in response to urine pressure (i.e., pressure sensitive).

In another aspect, a method for preventing urine backflow in a catheter is described. The method comprises the steps of inserting a catheter tube into the bladder of a user such that urine flows through the catheter tube, providing a urine backflow prevention device within the catheter tube, the device comprising a valve and a spring, enabling urine to push against the valve, causing the spring to compress and the valve to open, thereby enabling the urine to exit the catheter tube, and closing the valve when urine flow ceases, wherein the spring pushes the valve into a closed position, thereby preventing any remaining urine from flowing back into the bladder.

In still another aspect, the spring is made of stainless steel and is tightly coiled (i.e., coil spring) to provide the necessary resistance to keep the valve closed when no urine is flowing.

In another embodiment, a filter is disposed within the housing to prevent debris or small particles from entering the catheter tube.

In still another embodiment, the backflow prevention mechanism is positioned near the proximal end of the catheter tube to ensure efficient prevention of urine backflow into the bladder.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of a urine backflow prevention device of the present invention in accordance with the disclosed structure;

FIG. 2 illustrates a flow diagram depicting a process of preventing urine backflow using the urine backflow prevention device of the present invention in accordance with the disclosed structure; and

FIG. 3 illustrates a perspective view showing use of the urine backflow preventer device with the catheter tube in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an effective solution to prevent urine backflow in catheterized patients, which can lead to serious complications such as urinary tract infections (UTIs) and increased patient discomfort. There is also a long-felt need in the art for a reliable device that can be integrated into catheter systems to ensure that once urine is expelled from the bladder, it does not reenter the body, thereby maintaining the sterility of the urinary tract. Additionally, there is a long-felt need in the art for a catheter device that is simple to use, durable, and does not require frequent maintenance or replacement. Moreover, there is a long-felt need in the art for a device that can be easily sterilized or made disposable to further reduce the risk of infection. Furthermore, there is a long-felt need in the art for a urine backflow prevention system that can be used in a variety of medical settings, providing consistent performance and improving patient outcomes.

The present invention, in one exemplary embodiment, is a urine backflow prevention device. The device comprises a catheter tube configured to be inserted into a body to drain urine from a bladder, a housing, a valve is disposed within the housing, wherein the valve is configured to open in response to urine pressure (i.e., pressure sensitive) to enable urine to flow out of the catheter tube, and to close when urine flow ceases, and a spring is disposed within the housing and connected to the valve, the spring being calibrated to provide resistance sufficient to keep the valve closed in the absence of urine flow and to enable the valve to open when urine is expelled.

Reference will now be made in detail to the present preferred embodiments of the invention, examples of which are illustrated in the accompanying drawings. Wherever possible, the same reference numerals are used in the drawings and the description to refer to the same or like parts.

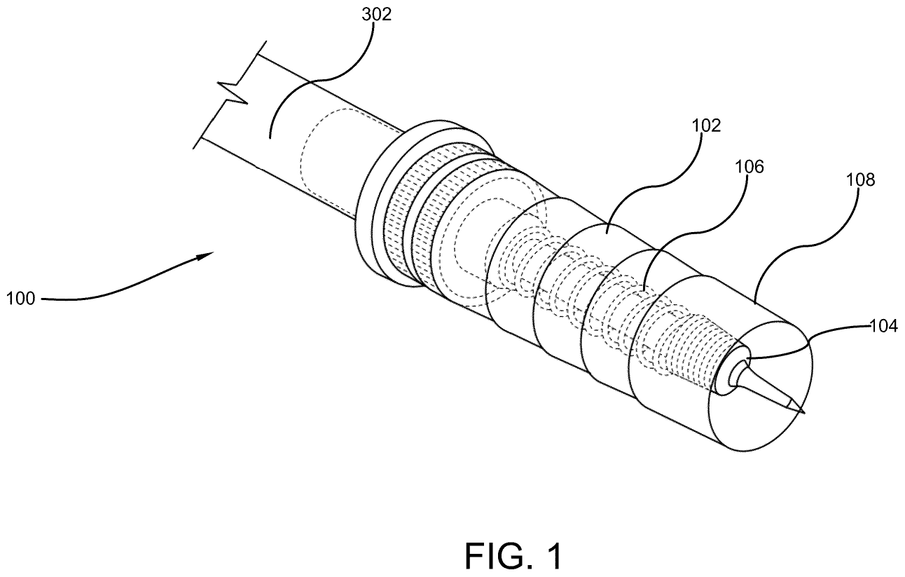

Referring initially to the drawings, FIG. 1 illustrates a perspective view of urine backflow prevention device of the present invention in accordance with the disclosed structure. The urine backflow prevention device 100 of the present invention is designed to prevent the backflow of expelled urine into the body. The urine backflow prevention device 100 is adapted to be used with catheters, which are tubes inserted into the body to drain urine from the bladder. More specifically, the backflow preventer device 100 is installed within a catheter tube 302 and is equipped with a small spring mechanism 102. The spring mechanism 102 is designed to respond to the flow of urine. When urine is expelled from the body (i.e., urinary tract) of a user, the urine flows past the backflow preventer device 100, pushing the device 100 open to enable the urine to exit. Once the flow of urine stops, the spring-loaded mechanism 102 automatically closes the valve, preventing any backflow.

As illustrated, the spring mechanism 102 includes a valve 104 which is preferably made from medical-grade plastic or rubber that is both durable and resistant to the chemical properties of urine. The valve 104 is preferably in the form of a circular disk or flap and is adapted to sit (i.e., be inserted) within a catheter tube 302. The valve 104 opens in response to urine pressure (i.e., pressure sensitive), enabling the urine to flow out. When urine stops flowing, the valve 104 closes to prevent any backflow into the bladder. In this manner the valve 104 functions as a one-way valve.

The spring-loaded valve urine backflow prevention device 100 also includes a spring 106 which can be made of stainless steel or another corrosion-resistant metal for reliability in a moist environment like the urinary tract. The spring 106 is small and tightly coiled (i.e., coil spring), thereby providing the necessary resistance to keep the valve 104 closed when there is no urine flow.

A housing 108 is integrated into the catheter tube 302 and is usually made of the same or compatible material as the tube 302. The housing 108 holds the spring 106 and valve mechanism 104 securely in place within the tube 302. The housing 108 provides a safe environment for the spring 106 and valve 104 enabling them to operate correctly every time urine flows through the catheter.

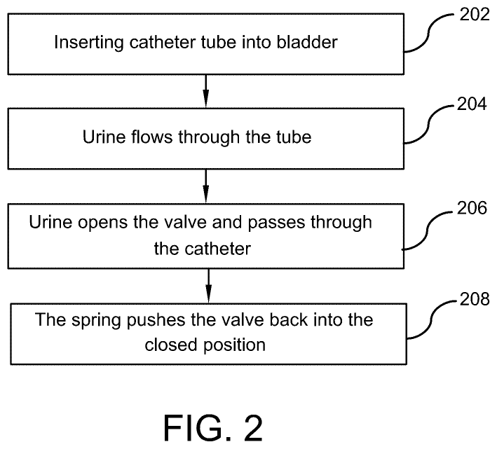

FIG. 2 illustrates a flow diagram depicting a process of preventing urine backflow using the urine backflow prevention device of the present invention in accordance with the disclosed structure. Initially, the catheter is inserted into bladder of a user such that urine of the user begins to flow naturally through the catheter tube 302 (Step 202). When urine flows through the tube 302, the urine pushes against the valve 104 within the spring-loaded backflow preventer device 100 (Step 204). The pressure from the urine compresses the spring 106 and opens the valve 104, thereby enabling urine to pass through the catheter and out of the body (Step 206). When the bladder of the user is empty and the flow of urine ceases, the spring 106 pushes the valve 104 back into the closed position thereof (Step 208). The closed valve prevents any urine remaining in the catheter or external environment from flowing back into the bladder, thus protecting against potential infections or other complications.

The spring-loaded valve urine backflow prevention device 100 is intended for medical use and can be easily sterilized or can be designed to be disposable after a single use to prevent infections. The device 100 does not clog or fail over time due to mineral deposits from urine. The device 100 reduces the risk of urinary tract infections (UTIs) by preventing expelled urine from reentering the body. The device 100 can also be helpful in surgical or post-operative settings where maintaining a sterile environment in the urinary tract is critical.

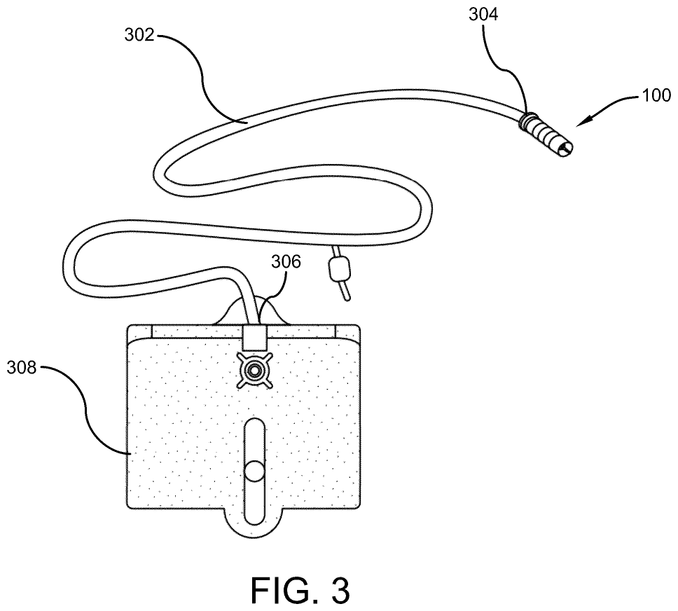

FIG. 3 illustrates a perspective view showing use of the urine backflow preventer device with the catheter tube 302 in accordance with the disclosed structure. The proximal end 304 of the catheter tube 302 is inserted into the bladder and may have additional features like an inflatable balloon (not shown) that helps keep the catheter in place inside the bladder. The opposite end 306 of the catheter 302 connects to a urine collection bag 308 or another external drainage system. The device 100 can be positioned at any appropriate place along the tube 302 but preferably near the proximal end 304 of the tube 302.

In some embodiments, a small filter may be included in the device 100 to prevent debris or small particles from entering the catheter and potentially clogging the device 100. It will be apparent to a person skilled in the art that the spring 106 within the backflow preventer device 100 is calibrated and sensitive to open when urine is expelled but strong enough to close immediately when the flow stops.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “urine backflow prevention device”, “spring-loaded valve urine backflow prevention device”, “urine backflow preventer device”, and “device” are interchangeable and refer to the spring-loaded valve urine backflow prevention device 100 of the present invention.

Notwithstanding the forgoing, the spring-loaded valve urine backflow prevention device 100 of the present invention can be of any suitable size and configuration as is known in the art without affecting the overall concept of the invention, provided that it accomplishes the above stated objectives. One of ordinary skill in the art will appreciate that the spring-loaded valve urine backflow prevention device 100 as shown in the FIGS. are for illustrative purposes only, and that many other sizes and shapes of the spring-loaded valve urine backflow prevention device 100 are well within the scope of the present disclosure. Although the dimensions of the spring-loaded valve urine backflow prevention device 100 are important design parameters for user convenience, the spring-loaded valve urine backflow prevention device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A urine backflow prevention device for catheter tubes comprising:
a housing;
a spring mechanism; and
a valve;
wherein said spring mechanism and said valve contained in said housing;
wherein urine flows past a backflow preventer device in one direction while being expelled from a user's urinary tract;
wherein said valve pushable in one direction to enable urine to flow past said backflow preventer device;
wherein said valve openable in said one direction in response to urine pressure; and
further wherein said spring mechanism closes said valve once flow of urine from the user's urinary tract stops.

2. The urine backflow prevention device for catheter tubes of claim 1, wherein said valve having a material selected from the group consisting of a medical-grade plastic and a rubber.

3. The urine backflow prevention device for catheter tubes of claim 2, wherein said valve is a circular disk.

4. The urine backflow prevention device for catheter tubes of claim 2, wherein said valve is a circular flap.

5. The urine backflow prevention device for catheter tubes of claim 3, wherein said valve and said spring mechanism insertable into a catheter tube.

6. The urine backflow prevention device for catheter tubes of claim 5, wherein said spring mechanism having a material of stainless steel.

7. The urine backflow prevention device for catheter tubes of claim 6, wherein said spring mechanism is a coil spring.

8. The urine backflow prevention device for catheter tubes of claim 7, wherein said housing integrated into the catheter tube.

9. The urine backflow prevention device for catheter tubes of claim 8, wherein the catheter tube and said housing having the same material.

10. A urine backflow prevention device for catheter tubes comprising:
a housing;
a spring mechanism; and
a valve;
wherein said spring mechanism and said valve contained in said housing;
wherein urine flows past a backflow preventer device in one direction while being expelled from a user's urinary tract;
wherein said valve pushable in one direction to enable urine to flow past said backflow preventer device;
wherein said valve openable in said one direction in response to urine pressure;
wherein said spring mechanism closes said valve once flow of urine from the user's urinary tract stops;
wherein the catheter tube and said housing having the same material; and
further wherein said spring mechanism is a coil spring.

11. The urine backflow prevention device for catheter tubes of claim 10, wherein said valve having a material selected from the group consisting of a medical-grade plastic and a rubber.

12. The urine backflow prevention device for catheter tubes of claim 10, wherein said valve is a circular disk.

13. The urine backflow prevention device for catheter tubes of claim 10, wherein said valve is a circular flap.

14. The urine backflow prevention device for catheter tubes of claim 10, wherein said valve and said spring mechanism insertable into a catheter tube.

15. The urine backflow prevention device for catheter tubes of claim 10, wherein said spring mechanism having a material of stainless steel.

16. The urine backflow prevention device for catheter tubes of claim 10, wherein said housing integrated into the catheter tube.

17. A method for preventing urine backflow within a catheter tube, the method comprising the steps of:
providing a housing, a spring mechanism, and a valve, wherein said spring mechanism and said valve contained in said housing, wherein the catheter tube and said housing having the same material, and further wherein said spring mechanism is a coil spring;
flowing urine past a backflow preventer device in one direction while being expelled from a user's urinary tract;
pushing said valve open in one direction to enable urine to flow past said backflow preventer device;
opening said valve in one direction in response to urine pressure; and
pushing said valve closed in another direction with said spring mechanism once flow of urine from the user's urinary tract stops.

18. The method for preventing urine backflow within a catheter tube of claim 17, wherein said valve having a material selected from the group consisting of a medical-grade plastic and a rubber.

19. The method for preventing urine backflow within a catheter tube of claim 18, wherein said valve is a circular flap.

20. The method for preventing urine backflow within a catheter tube of claim 19, wherein said spring mechanism having a material of stainless steel, and further wherein said housing integrated into the catheter tube.
